(19) United States
(12) Patent Application Publication
(10) Pub. No.: US 2005/0171569 A1
(43) Pub. Date: Aug. 4, 2005

(54) Method and apparatus for creating intrauterine adhesions

(75) Inventors: Michael J. Girard, Lino Lakes, MN (US); Paul Danielson, Shakopee, MN (US); James Elliott Coad, Morgantown, WV (US); James Presthus, Edina, MN (US); Karen Elizabeth Peterson, Eagan, MN (US); Douglas J. Duchon, Chanhassen, MN (US)

Correspondence Address: INSKEEP INTELLECTUAL PROPERTY GROUP, INC, 1225 W. 190TH STREET, SUITE 205, GARDENA, CA, 90248, US

(73) Assignee: ImPres Medical, Inc.

(21) Appl. No.: 10/978,176

(22) Filed: Oct. 29, 2004

Related U.S. Application Data
(63) Continuation-in-part of application No. 10/726,433, filed on Dec. 3, 2003.
Application No. 10/726,433 is a continuation of application No. 09/840,951, filed on Apr. 24, 2001, now Pat. No. 6,708,056.
(63) Continuation-in-part of application No. 10/851,364, filed on May 21, 2004.
(63) Continuation-in-part of application No. 10/850,761, filed on May 21, 2004.
(60) Provisional application No. 60/256,529, filed on Dec. 18, 2000.
(60) Provisional application No. 60/199,736, filed on Apr. 25, 2000.
(60) Provisional application No. 60/472,643, filed on May 21, 2003.
(60) Provisional application No. 60/472,644, filed on May 21, 2003.

(51) Int. Cl. A61F 2/06; A61B 18/18; A61M 29/00

(52) U.S. Cl. 606/193; 623/1.15

(57) ABSTRACT

In general, the present invention contemplates an implantable device for treating excessive bleeding in a body cavity. The device comprises a biocompatible material, for example polyethylene teraphathalate (PET), which is deliverable into the body cavity. The biocompatible material contains an attribute(s) that promotes tissue reaction or growth that results in a tissue response and/or adhesion formation within the body cavity to reduce or stop the excessive bleeding.

### RELATED APPLICATIONS

This application is a continuation-in-part of U.S. application Ser. No. 10/726,433 filed Dec. 3, 2003 entitled Method And Apparatus For Creating Intrauterine Adhesions which is a continuation of U.S. application Ser. No. 09/840,951 filed Apr. 24, 2001 entitled Method And Apparatus For Creating Intrauterine Adhesions now U.S. Pat. No. 6,708,056 which is a non-provisional application claiming priority to U.S. Provisional Application Ser. No. 60/256,529 filed Dec. 18, 2000 and U.S. Provisional Application Ser. No. 60/199,736 filed Apr. 25, 2000, both of which are now abandoned.

This application is also a continuation-in-part of U.S. application Ser. No. 10/851,364 entitled Bioreactive Methods and Device for Abnormal Bleeding filed May 21, 2004 which is a non-provisional application claiming priority to U.S. Provisional Application Ser. No. 60/472,643 filed May 21, 2003.

This application is also a continuation-in-part of U.S. application Ser. No. 10/850,761 entitled Intrauterine Implant and Methods of Use filed May 21, 2004 which is a non-provisional application claiming priority to U.S. Provisional Application Ser. No. 60/472,644 filed May 21, 2003.

All of the above applications are incorporated herein by reference.

### BACKGROUND OF THE INVENTION

Menstrual bleeding is a part of normal life for women. The onset of menstruation, termed menarche, usually occurs at the age of 12 or 13. The length of a woman's monthly cycle may be irregular during her first one to two years. Once the menstrual cycle stabilizes, a normal cycle may range from 20 to 40 days and on average lasts 28 days. Age, weight, athletic activity and alcohol consumption are several factors that can affect menstrual cycles. For example, younger women (under the age of 21) and older women (over the age of 49) tend to have longer cycle times, generally averaging 31 days and over. Similarly, women who are very thin or athletic may have longer cycles. In contrast, women who consume alcohol on a regular basis tend to have shorter cycle times.

Nearly all women, at some time during their reproductive life, experience some form of menstrual irregularity or abnormality. These disorders range from mild to severe, often resulting in numerous lost work hours and the disruption of personal/family life each month. In general, physical symptoms such as bloating, breast tenderness, severe cramping (dysmenorrhea) and slight temporary weight gain frequently occur during most menstrual cycles. In addition, emotional hypersensitivity is also common, including depression, anxiety, anger, tension and irritability. These symptoms are generally worse a week or so before a woman's menstrual period and resolve afterward.

Many women also suffer from a condition called menorrhagia (heavy bleeding). Menorrhagia is a clinical problem characterized by substantial discomfort and heavy flow/bleeding, characterized by blood loss exceeding 80 cc/month. It is estimated that 1 in 5 women between the ages of 35 and 50, or approximately 6.4 million women in the United States alone, are affected by menorrhagia. Fibroids, hormonal imbalance and certain drugs, such as anticoagulants and anti-inflammatory medications, are common causes of heavy bleeding.

Women diagnosed with menorrhagia or dysmenorrhea have limited treatment options available to them. Currently, other than hormone therapy and a few experimental pain management techniques, hysterectomy (removal of the uterus) and endometrial ablation/resection (destruction of the uterine lining) are the clinically accepted treatment modalities for menorrhagia. These surgical procedures either eliminate or substantially reduce the possibility of childbearing. Further, hysterectomy requires up to a six-week recovery time following surgery and commonly a lifetime of hormone therapy when the ovaries are also removed. Endometrial ablation has a low success rate at achieving amenorrhea (cessation of menstrual bleeding). As a result, many of the women affected by menorrhagia are driven to make lifestyle-altering decisions.

Since the 1800's, attempts using various treatments have been made to control uterine bleeding by means other than hysterectomy. Alternative methods include chemicals, steam, ionizing radiation, lasers, electrocautery, cryosurgery and others. The long-term risk for some of these methods can be quite high and may lead to other more serious complications such as mesodermal tumors or uterine cancer.

Clinically, a condition known as Asherman's syndrome has been observed where adhesions within the uterine cavity disrupt the normal menstrual cycle. This leads to a reduction in bleeding from the normal menstrual cycle and often produces amenorrhea.

In 1894, Heinrich Fritsch was the first to describe amenorrhea resulting from traumatic obliteration of the uterine cavity following puerperal curettage. However, it was not until the late 1940's that knowledge about its association with uterine adhesions (synechiae) was first disseminated in medical journals by Joseph G. Asherman, for whom the condition is named. In 1957, the 17th Congress of the Federation of French Speaking Societies of Gynecology and Obstetrics proposed the following classification of uterine synechiae:

Traumatic Synechiae connected with surgical or obstetrical evacuation of the uterus;

Spontaneous synechiae of tuberculosis origin;

Synechiae occurring after myomectomy; and

Synechiae secondary to chemical or physical agents and likewise those resulting from atrophic changes.

In general, two types of traumatic synechiae are currently recognized. The first type is stenosis or obliteration of the endocervical canal. The second type of traumatic synechiae is partial or complete obliteration of the uterine cavity by conglutination of the opposing walls.

Other terms, such as endometrial sclerosis, traumatic uterine atrophy, uterine artesia, uterine synechiae and adhesive endometriosis, have also been used to describe the phenomena of Asherman's Syndrome. The severity of adhesion is generally classified into one of the following three groups or classes: Class I represents adhesions occurring in less than one-third of the uterine cavity with both ostia (i.e. openings of the fallopian tubes) visible; Class II represents adhesions occurring in one-third to one-half of the uterine cavity with one ostium visible; and Class III represents adhesions occurring in greater than one-half of the uterine cavity with no ostia visible.

Although Asherman's Syndrome has been studied extensively and numerous articles and papers have been written on the topic, uncertainty still exists as to the predominant causative factor(s) and biological mechanism(s). It is believed that if the endometrium is severely damaged, it may be replaced by granulation tissue. When this happens, the opposing uterine walls adhere to one another and form scar tissue. In particular, adhesions form and transluminally bridge the anterior and posterior surfaces of the uterus. The adhesions or tissue that is formed between the walls comprises connective tissue that is, typically, avascular. Soon after, the tissue may be infiltrated by myometrial cells and, later, covered by endometrium.

Conventionally, intrauterine adhesions have been regarded as undesirable conditions (for example U.S. Pat. No. 6,211,217, issued to Spinale et al, U.S. Pat. No. 6,136,333, issued to Cohn et al. and U.S. Pat. No. 6,090,997, issued to Goldbert et al.). Indeed, in several known treatment methods for menorrhagia, it has been encouraged to avoid the creation of adhesions. Even in those circumstances where clinicians have experimented with adhesion formation, the results have not proved promising. For example, in the March 1977 edition of the Israel Journal of Medicine, an article by J. G. Schenker, entitled Induction of Intrauterine Adhesions in Experimental Animals and Women, described an experiment in which surgical sponges were implanted into the subcutaneous wall of the patient. The sponges remained in the subcutaneous wall until fibroblasts, or connective-tissue cells, populated the sponges. Next, the sponges were removed and implanted into the uterus of the same patient.

Schenker observed that, after a period of time, adhesions were formed in the areas adjacent to the location of the implanted fibroblast bearing sponge. No adhesions were observed in areas that did not have contact with the fibroblast bearing sponge. These experiments were carried out in several animal models (for example, rabbit, rat and primates) and humans. Schenker concluded that it was possible to artificially create adhesions within the uterus, but that such a procedure was not practical.

In U.S. Pat. No. 6,708,056 issued to Duchon et al., the contents of which are hereby incorporated by reference, a method for creating intrauterine adhesions resulting in amenorrhea was presented, including devices for creating such intrauterine adhesions. Specifically, Duchon et al. contemplated an implantable device comprised of biocompatible material which promotes tissue growth, resulting in adhesion formation.

Although adhesion formation remains one method of inducing amenorrhea, the inventors have discovered other methods for potentially obtaining a reduction in bleeding that preferably produces amenorrhea. For example, these methods may cause the complete replacement of the uterine functionalis/basalis endometrium, creating blockage of the uterine endocervical canal, creating a discrete architectural change of the uterine cavity and/or others are also believed to result in amenorrhea or at least a reduction in menstrual bleeding.

What are needed are improved methods and devices to take advantage of these newly understood mechanisms of action, as well as improved methods and devices for the previously discovered mechanisms. All of these items are to provide better treatment for abnormal uterine bleeding.

### OBJECTS AND SUMMARY OF THE INVENTION

Therefore, it is an object of the present invention to overcome the limitations of prior treatments of excessive bleeding within a body cavity.

It is an object of the present invention to provide an implantable device for treating excessive bleeding in a body cavity.

It is another object of the present invention to provide a method of pretreating a uterus to better treat excessive bleeding.

In general, the present invention contemplates an implantable device for treating excessive bleeding in a body cavity. The device comprises a biocompatible material, for example, polyethylene teraphathalate (PET), which is deliverable into the body cavity. The biocompatible material contains an attribute that promotes tissue growth that results in adhesion formation within the body cavity.

The present invention also contemplates a method of creating adhesions in a body cavity. In general, the method comprises inserting an implantable device within the body cavity. The method also includes locating the implantable device at an optimal site within the body cavity, wherein the optimal site promotes effective adhesion formation for controlling bleeding.

The present invention also contemplates a method and devices for treating excessive bleeding within a body cavity without creating adhesions.

The present invention further contemplates a pretreatment method for creating trauma to a tissue within a body cavity.

### BRIEF DESCRIPTION OF THE DRAWINGS

FIG. 1 illustrates a front view of a T-shaped implant according to the present invention;

FIG. 2 illustrates a front view of a T-shaped member according to the present invention;

FIG. 3 illustrates a front view of the implant of FIG. 1 within a uterus;

FIG. 4 illustrates a front view of a V-shaped implant according to the present invention;

FIG. 5 illustrates a front view of another embodiment of V-shaped implant according to the present invention;

FIG. 6 illustrates a front view of a T-shaped implant according to the present invention;

FIG. 7 illustrates a side view of the T-shaped implant of FIG. 6;

FIG. 8 illustrates a view of a uterine implant with bridging member according to the present invention;

FIG. 9 illustrate a front view of rolled implants according to the present invention;

FIG. 10 illustrates a front view of a T-shaped implant with rolled fabric according to the present invention;

FIG. 11 illustrates flat fiber strands according to the present invention;

FIG. 12 illustrates textured fiber strands according to the present invention;

FIG. 13 illustrates a perspective view of a lower uterine segment endocervical implant according to the present invention;

FIG. 14A illustrates a front view of a portion of an implant with an open channel construction according to the present invention;

FIG. 14B illustrates a cross-section view of the implant with open channel construction of FIG. 14A;

FIG. 14C illustrates a cross-section view of the implant with multiple layers of fabric;

FIG. 15 illustrates fabric ball implants inserted according to the present invention;

FIG. 16 illustrates a side view of a ring implant according to the present invention;

FIG. 17 illustrates a front view of a tissue penetrating implant according to the present invention;

FIG. 18 illustrates a view of the tissue penetrating portion of the implant in FIG. 17;

FIG. 19 illustrates a side view of a tube strut according to the present invention;

FIG. 20 illustrates a side view of an alternate embodiment of the strut shown in FIG. 19 according to the present invention;

FIG. 21 illustrates a cross-section view of a tissue penetrating implant according to the present invention;

FIG. 22 illustrates a perspective view of a tissue penetrating implant according to the present invention;

FIG. 23 illustrates a side cross-section view of the placement/delivery of the tissue penetrating implant of FIG. 22;

FIG. 24 illustrates a midline cross-section view of the tissue penetrating implant of FIG. 22 as deployed within a uterus;

FIG. 25 illustrates a front sectional view of another embodiment of a tissue penetrating implant deployment within a uterus according to the present invention;

FIG. 26 illustrates a front view of another embodiment of a tissue penetrating implant according to the present invention;

FIG. 27 illustrates a side view of the tissue penetrating implant of FIG. 26;

FIG. 28 illustrates a front view of a fan-like implant according to the present invention;

FIG. 29 illustrates a cross-section view of an implant within the uterus according to the present invention;

FIG. 30A illustrates a front view of another embodiment of tissue penetrating implants according to the present invention;

FIG. 30B illustrates a front view of the tissue penetrating implants of FIG. 30A;

FIG. 30C illustrates a front view of a tissue penetrating implant according to the present invention;

FIG. 31 illustrates a cross-section view of a barbed connector implant according to the present invention;

FIG. 32 illustrates a front bi-valved view of a continuous fiber implant within the uterus according to the present invention; and,

FIGS. 33A-33C illustrate front views of a stentless implant and method of deploying the same in accordance with a preferred embodiment of the present invention.

### DETAILED DESCRIPTION OF THE INVENTION

Mechanisms of Action

Excessive menstrual flow or bleeding, termed menorrhagia, is indicative of abnormal sloughing of the endometrial tissue layer. Unlike conventional therapies such as hysterectomy or ablation/resection procedures, the embodiments of the present invention achieve reduced bleeding with the intended outcome of reduced bleeding or amenorrhea (cessation of bleeding) by way of an implant, which decreases or deactivates the endometrial tissue. There may be several contributing mechanisms or steps of action to create amenorrhea or reduced bleeding in a patient. Many of these mechanisms may or may not relate to the creation of adhesions, as non-adhesion based mechanisms of action may also achieve clinical amenorrhea to eliminate abnormal uterine bleeding.

One such mechanism, previously discussed in U.S. Pat. No. 6,708,056, involves the complete obliteration of the uterine cavity. Specifically, this involves replacement of the uterine endometrium and the adjacent virtual intrauterine cavity space with another tissue or substance such as would form an adhesion or other contact of the cavity wall(s). This adhesion may consist of fibrous - granulation tissue, extracellular matrix (e.g. collagen) or any other durable tissue (e.g. endometrial stromal tissue). Such tissue growths may be induced by an implanted material or device that creates a tissue response, an induced tissue trauma or a combination of both (trauma with response). Since all of the endometrial tissue with regenerative cycling potential has been replaced by a non-shedding or noncycling material or tissue, amenorrhea or at least reduced bleeding is thus induced. Additionally, the uterine cavity is completely obliterated, and the concept should result in infertility or an effective contraceptive. Further, the potential for endometrial cancer should be reduced since the endometrial tissue is either reduced, inactivated or eliminated.

Another mechanism, similar to the previously described mechanism, involves obliteration of all or a portion of the endometrium, followed by replacement of the endometrium by another cellular tissue substance or extracellular matrix (e.g. collagen). However, unlike what may occur in the previously described mechanism, the opposing walls of the uterine cavity are not completely adhered together. Similarly, the tissue response may be induced by an implanted material or device, induced trauma, or both. However, if an implant device is utilized, it would be composed of material(s) that creates a barrier to limit tissue in-growth to prevent the opposing uterine walls from adhering together. Therefore, an intrauterine cavity is maintained within the barrier protected portion of the implant, allowing for continued access to the intrauterine cavity for diagnostic purposes and to prevent fluid collections, such as hematometra (retention of blood within the uterus).

Yet another mechanism of amenorrhea involves creating a complete blockage, closure or obstruction of the intrauterine cavity anywhere in the lower uterine segment to the internal endocervical os, preventing menstrual blood or other fluid from escaping or draining from the uterine cavity. Although the endometrium still exists in the remaining cavity above the obstruction, menstruation stops, generally without resulting in hematometra. The inability of the shedding endometrium to pass or drain from the cavity may inactivate the endometrium so that it no longer cycles, thus creating amenorrhea without hematometra. The complete blockage of uterine outflow may be produced by tissue adhesion(s) or growth that is induced by an implanted material or device that creates a tissue response, an induced trauma or a combination of both. Such an obstruction or closure may consist of fibrous granulation tissue, extracellular matrix (i.e. collagen) or any other durable tissue such as endometrial stromal tissue. Additionally, the cervical blockage may include and/or result from mechanical devices that cause endocervical blockage.

Another mechanism of amenorrhea involves creating discrete architectural changes of the uterine tissues and/or its cavity, such as creating connections between opposing uterine walls. These connections may be produced by creating adhesion(s) in a small uterine cavity region instead of involving the complete cavity and may consist of fibrous granulation tissue, extracellular matrix (i.e. collagen) or any other durable tissue such as endometrial stromal tissue. The adhesion(s) may result from an implanted material or device that creates a tissue response, an induced trauma or a combination of both. Alternatively, the architectural change may also be effectively produced with a mechanical connection that does not rely on adhesion formation.

Amenorrhea may result in these contexts by several possible mechanisms, either singularly or in concert with other signaling or mechanical pathways. For example, the adhesions or mechanical connections may alter signaling or disrupt the peripheral nerve system associated with the uterus with the net effect of variable endometrial inactivation. For example, a neural change could produce an involuntary reflex of the lower uterine segment or internal endocervical os muscles, resulting in out flow occlusion of the cavity. In another example, midline adhesion(s) or mechanical connection(s) between the uterine walls may alter the mechanics of the uterine muscle or cavity preventing normal menstrual cycling. In yet another example mechanism, the endometrium surrounding the adhesion does not develop to full functionalis thickness, possibly due to a maturation arrest that prevents the endometrium from completing its cycle and halting the menstrual cycle.

Another mechanism of amenorrhea involves insertion of a device within the uterine cavity that creates contact with the endometrial tissue(s) without creating adhesions. Such changes may be the result of direct contact pressure from the device on the endometrium or may be the result of cellular signaling changes when contact between the endometrial surfaces lack direct contact secondary to the device.

In regard to the previously described mechanisms of amenorrhea, multiple implant device embodiments are herein described according to the present invention to take advantage of these possible mechanisms. Others have been described in copending U.S. application Ser. No. 10/850,761 filed May 21, 2004 entitled Intrauterine Implant and Methods of Use, the entire contents of which are incorporated herein by reference. Generally, many of the embodiments described in this application utilize various forms of polyethylene teraphathalate (PET), also commonly referred to as polyester. Dacron is the trade name for one commonly produced PET material. The invention (including the embodiments described) may be composed of other biocompatible materials alone or in combination with PET or other similar materials.

PET is a preferred uterine implant material due to its ability to elicit a tissue response, which is bioreactive in nature, followed by a fibrotic tissue incorporation or reaction. This response is discussed in detail in co-pending U.S. application Ser. No. 10/851,364 filed May 21, 2004, entitled Bioreactive Methods and Devices for Treating Abnormal Bleeding, the entire contents of which are hereby incorporated by reference. In addition to eliciting a tissue response, PET may be used as a scaffolding to support, enhance and/or control the primary tissue in-growth and potentially combine with another biocompatible material that creates tissue trauma or specific tissue access. Coatings such as collagen, TGF-beta, hormones (such as progesterone) or other stimulants that may enhance or accelerate a tissue response are also contemplated by this invention. For the purposes of this application, a bioreactive material is intended to refer to all types of materials that are not only biocompatible but also a material that causes a biological response in the body. In a preferred embodiment, a bioreactive material is one where the biological response is a fibrotic or other durable tissue response.

PET filaments or fibers are the individual elements that make up a yarn bundle. The filaments can be in different shapes such as round, oval, tri-lobal or others. The size of the filaments is measured in Denier, a textile term. One denier is approximately equal to 10 micrometers or 0.0004 inches in diameter. For example, a preferred fiber size is between about 1 and 20 denier. For a specific amount of material, a fine filament denier with more filaments in the yarn bundle may be preferred over a higher denier filament with fewer filaments, due to the increased surface area the former option provides (in which case it is preferred that the filaments are loosely arranged in the yarn bundle). The increased surface area has the potential to increase tissue exposure and thus may provide a better tissue response. Additionally, multifilament yarn configurations are generally more compliant and conforming than monofilament configurations of similar Denier and material type.

Yarns can have a flat surface, as seen in FIG. 11 or textured, as seen in FIG. 12. A highly textured yarn is typically preferred over flat yarn due to increased porosity and surface area for tissue interaction. Further, yarn texture also influences the surface roughness of any fabric construction(s) of the uterine implant.

The fabric construction determines the macro porous configuration of PET or other fabrics potentially used with the implant. Such a fabric may be knit, woven, or non-woven. A knit mesh construction or a non-woven needle felt are two example fabric constructions preferred to provide an open scaffold to encourage tissue in-growth after being evaluated in animal testing. Fibrous tissue in-growth is enhanced by pore sizes of at least about 50-250 micrometers and preferably a pore size in the range of 100 to 200 micrometers. Fabric constructions that provide open channels between layers also may promote tissue proliferation. FIG. 14C shows a layered fabric 161 with parallel running spacers 165 that provide separation between the fabric layers, resulting in open channels 163 for tissue in-growth and propagation. FIGS. 14A and 14B show a similar fabric however there is no second layer and the channels 163 are open.

T-Shaped Implant Device

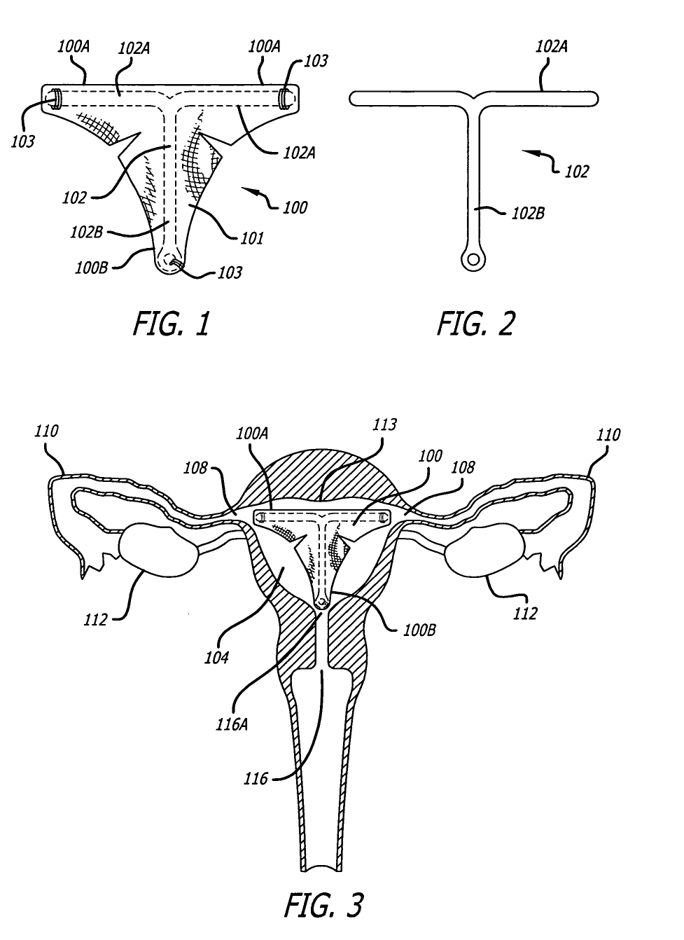

Referring to FIGS. 1-3, a preferred embodiment of the present invention illustrates a T-shaped kite-like uterine implant 100. The design was implemented utilizing approved clinical implant components to gain initial clinical experience. Generally, a layer of PET fabric 101 is fixed by sutures 103 to a T-shaped structure 102 having two arms 102a and a body 102b, as seen best in FIG. 2. Once implanted, the PET fabric stimulates a tissue response to induce an adhesion(s) to and/or between the uterine walls, thus reducing bleeding and optimally inducing amenorrhea.

Generally, the T-shaped structure has two arms 102a and a single elongated body 102b. Further, the T-shaped structure 102 provides a semi-rigid deploying structure for the PET fabric 101, which impedes the T-shaped uterine implant from being ejected by uterine contractile forces. Once implanted within the uterus 104, the PET fabric 101 promotes the formation of intrauterine adhesions, thus reducing and/or deactivating the endometrial tissue and reducing or eliminating the bleeding.

FIG. 3 illustrates the T-shaped uterine implant 100 positioned within the uterus 104 of a patient. Two openings 108 at the top of the uterus 104 lead to the fallopian tubes 110 and ovaries 112, while a lower opening of the uterus 104 is formed by the cervix 116. The walls of the uterus 104 are generally composed of three layers of tissue: The inner endometrium, the middle myometrium and the outer perimetrium/serosa. It is the inner endometrial layer or lining that separates/breakdowns within the uterus 104 and leaves the body as the menstrual flow during a woman's menstrual period.

In one specific example of the T-shaped uterine implant 100, a Mirena Intra Uterine Device (IUD) stent may be used as the T-shaped structure 102 (once the hormone cylinder is removed) and a thin layer of Bard DeBakey Double Velour PET (Style #6110) cardiovascular fabric may be used as the PET fabric, fastened by polypropylene sutures 103 to the T-shaped structure 102. The T-shaped uterine implant 100 may then be positioned within the uterus 104 of a patient using a simple delivery tube cannula (e.g. 6 mmlD) and a stylet (e.g. pusher rod). The delivery tube allows the uterine implant 100 to be packed within the tube and pushed out within the intrauterine cavity 104 in the desired treatment location, preferably positioned similarly to a typical Mirena IUD. Preferably, the T-shaped implant 100 is implanted within a patient, causing a fibroproliferative, stromal or other tissue response within the uterus 104 that leads to adhesion(s), resulting in reduced bleeding or preferably amenorrhea, similar to what is observed clinically with Asherman's syndrome. The device described has been implanted in patients and followed for a period of 30 to 90 days to observe fibroproliferative and stromal tissue responses.

In practice, the narrow end 100b of the T-shaped uterine implant 100 is first loaded into a cannula sheath (not shown), while the arms 100a are folded up away from the narrow end 100b and towards each other. The T-shaped uterine implant 100 is then completely loaded into the distal end of the cannula sheath (patient end) while a stylet (not shown) is introduced into the proximal portion of the cannula sheath until it abuts with the implant tip. Next, minimum cervical dilation of approximately 6.5 mm is achieved and, if desired, uterine cavity pretreatment can be performed. Pre-treatment procedures such as resection, endometrial ablation, dilation and curettage or others may be used prior to implantation of the T-shaped uterine implant 100. Further details of example pre-treatment procedures are described elsewhere in this application. Next, the cannula is introduced into the endocervical canal through the exocervical os with gentle forward advancement until it reaches the superior fundic wall 113 of the intrauterine cavity. The cannula is then retracted 2 cm so that the proximal tip of the cannula is 2 cm from the superior fundic wall 113. Next, the stylet is advanced approximately 1.5 cm while holding the cannula sheath in place, allowing deployment of the arms 100a while leaving the body 100b within the cannula. The stylet and cannula sheath are then readvanced to the superior fundic wall to position the arms 100a in the desired extended position with the arm tips extended towards the cornu. The stylet is then held in place as the cannula sheath is retracted, deploying the implant body 100b. Finally, the stylet may be withdrawn from the patient, leaving the T-shaped uterine implant 100 within the intrauterine cavity approximately as shown in FIG. 3.

FIGS. 6 and 7 illustrate a similar alternate preferred embodiment of a uterine implant 132 according to the present invention. As with the T-shaped uterine implant 100, the present uterine implant 132 includes a T-shaped structure 102, seen in FIG. 2, such as a Mirena IUD stent with the hormone cylinder removed. However, the uterine implant 132 includes fabric 134 more closely shaped to the inner contours of the uterine cavity 104 and also having an improved macrostructure to better promote tissue in-growth. Specifically, for example, the fabric 134 is comprised of a double layer of Bard DeBakey Elastic Knit PET (style # 6106) cardiovascular fabric that encapsulates or covers both sides of the T-shaped structure. The fabric is sewn together along its edge using 4-0 braided PET suture 131. In addition, the fabric 134 has apertures 136, preferably positioned randomly along the fabric 134 at about 2 mm or 5 mm spacing, except near the T-shaped structure 102. Preferably, the apertures are 1/16 inch in diameter, allowing tissue growth through the fabric 134 and ultimately creating bridging adhesions between the walls of the uterus 104. This preferred embodiment may be implanted using a similar procedure as described previously and may preferably include pretreatment procedures consisting of Zoladex, birth control pills, resection, dilation and curettage or others. Further details of example pre-treatment procedures are described elsewhere in this application. Although the DeBakey Elastic Knit PET has been implemented in practice, this invention contemplates and considers other potentially more optimal fabric constructions for use with the T-shaped structure or other possible support frame configurations.

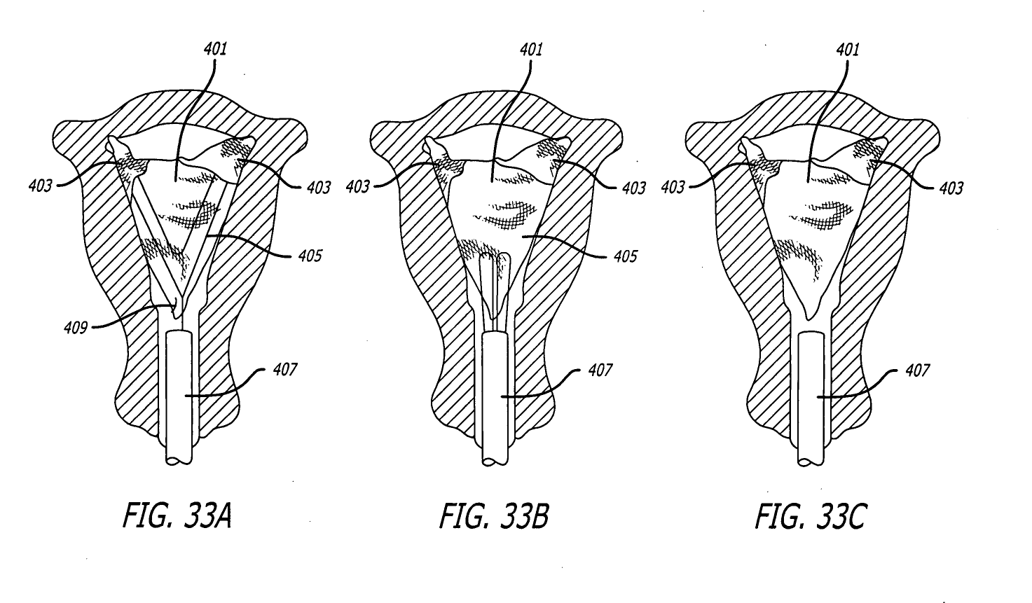

In an embodiment related to the implant of FIGS. 6-8, reference is made to FIGS. 33A-33C. In this embodiment, the implant as deployed in the uterine cavity is “stentless” meaning there is no T-shaped structure supporting the fabric as there is in the embodiment of FIGS. 6 and 7 (i.e., there is no structure such as the Mirena IUD stent with the hormone cylinder removed as shown in FIGS. 6 and 7). This becomes more clear with reference to the FIGS. 33A-33C.

As shown in FIG. 33A, the implant 401 is comprised of a triangularly shaped fabric material with two pockets 403 formed in the upper opposing corners of the fabric. During deployment, the two pockets 403 receive two ends of a deployment stent 405, the deployment stent 405 being extendable and retractable from a delivery cannula 407. Once the delivery cannula 407 has been inserted into the opening of the uterus as shown, the deployment stent 405 is advanced with slight pressure to move the implant 401 toward the fundic wall. As the deployment stent 405 advances, the two ends of the deployment stent 405 located in the pockets 403 expand away from each other and thereby expand the implant 401 into conformance with the shape of the uterine cavity.

At the same time the deployment stent 405 is being urged toward the fundic wall, a suture 409 that is attached at one end to the lower portion of the implant 401 is held in tension by the user so as to ensure that the fabric of the implant 401 is fully expanded and to ensure that it lays flat with within the uterine cavity. Once this has been achieved, the suture 409 is then cut and removed from the fabric of the implant 401.

Once the suture 409 has been cut and removed from the fabric of the implant 401, the user begins to retract the deployment stent 405 back into the cannula 407 as shown in FIG. 33B. As is seen, the opposing ends of the deployment stent 405 are urged closer together again so that the deployment stent 405 can fit back into the cannula 407.

Referring to FIG. 33C, once the deployment stent 405 has been fully retracted into the cannula 407, the cannula 407 can then be removed from the uterus. The implant 401 then remains located in the uterine cavity as shown in FIG. 33C.

FIG. 8 illustrates another preferred embodiment of a uterine implant 140. In addition to the fabric 146, preferably made from PET, the present invention includes bilateral fallopian tube extensions 142 and an endocervical canal extension 144. The fallopian tube extensions 142 are a semi-rigid structure located near the sides of the wide portion of uterine implant 140. When implanted in a uterus 104, the fallopian tube extensions 142 extend on both sides into the openings 108 of the fallopian tubes 110. The endocervical extension 144 is a similar semi-rigid structure located near the narrow end of the uterine implant 140 that extends into the endocervical canal 116.

In addition to creating the previously described tissue response within the uterus 104 with fabric 146, the uterine implant 140 may illicit or draw on a more robust fibrosis healing response from fallopian tubes 108 and/or the endocervix 116. The implant would facilitate drawing the cells necessary for a fibroproliferative response into the main cavity. Fibrosis and adhesions within the uterus 104 are sometimes difficult to create without trauma and/or contact with the tissue lying below the endometrium. The normal cycling of the endometrium, which lines the cavity, may prevent or inhibit the pathways/mechanisms related to generating a fibroproliferative response similar to that observed in other parts of the body. However, the mucosal tissues are biologically different in the endocervix 116 and fallopian tubes 110 than the uterus 104, since they do not cycle and regularly regenerate their mucosal layer. Thus, the fallopian tubes and endocervical regions are potentially more receptive for inducing a fibroproliferative response. In this respect, the extension 142, 144 may enhance initiation and propagation of this tissue response by inducing a trauma or a foreign body response. The fibrosis caused by the extensions 142, 144 may effectively spread across the uterine implant 140, which acts as a scaffold for the tissue. The resulting mass of fibrosis tissue may result in additional pressure within the uterus 104 or possibly replacement of the endometrium, but in either case bleeding is reduced and amenorrhea is preferably obtained.

V-Shaped Implant Device

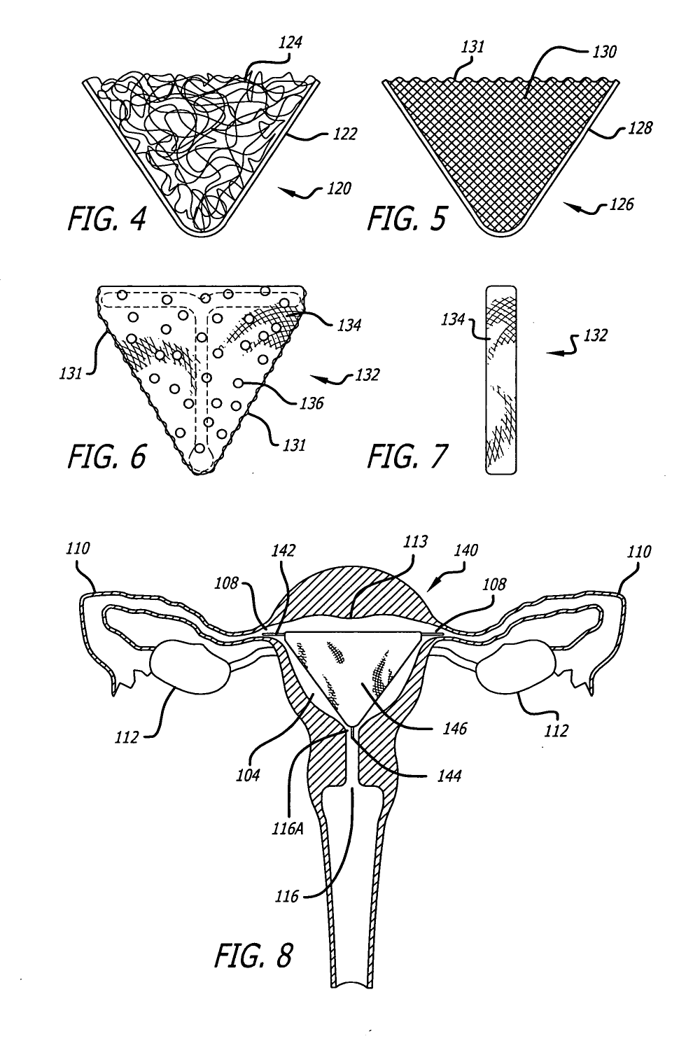

Referring to FIG. 4, a V-shaped yarn implant 120 is illustrated according to the present invention. The V-shaped yarn implant 120 has a semi-rigid V-shaped member 122 which acts as a frame for randomly oriented yarn fibers 124. By utilizing an overall V shape with curved ends, the V-shaped yarn implant 120 closely matches the funnel shape of the intrauterine cavity 104.

The V-shaped member 122 is preferably composed of a semi-rigid material that can flex under strains and gently conform along the funneled lateral walls of the uterus, yet is rigid enough to prevent ejection by uterine contractile forces. Simple calculations of stiffness for the material may be used to determine a desired V-shaped member 122 diameter. The stiffness, S of the V-shaped member 122 is proportional to 3EI/L³, where E represents the elastic modulus of the material, I represents the second moment of area, and L represents the length. For example, the V-shaped member 122 may be composed of a nitinol wire 0.020 to 0.025 inches in diameter. Thus, the V-shaped member 122 may flex when positioned within a cannula for deployment within a uterus 104. Preferred example dimensions of the V-shaped member 122 include a maximum width of the V shape of about 3.2 cm and a height of about 4.2 cm to match the size and shape of a typical uterus.

The randomly oriented yarn fibers 124 are fixed to the V-shaped member 122, filling out the space directly between the V of the member 122. Thus, once implanted into the uterus 104, the randomly oriented yarn fibers 124 contact nearly the entire uterine cavity and stimulate a tissue response. PET textured multifilament yarn is preferred, having a preferable minimum pore size of about 50 to 250 micrometers between yarn fibers. The open structure created by the randomly oriented yarn fibers 124 provide a scaffold to promote tissue in-growth within the device that can result in adhesions between the uterine walls.

In operation, the V-shaped yarn implant 120 is preferably implanted within the uterus 104 of a patient with a cannula (not shown) or delivery tube, similar to above, which allows the V-shaped yarn implant 120 to be compressed and positioned within the uterus 104. The V-shaped yarn implant 120 deploys within the uterus 104 to match its overall funnel shape, providing maximum contact between the uterine walls and the yarn fibers 124.

FIG. 5 illustrates a similar alternate preferred embodiment according to the present invention. The V-shaped implant 126 has an overall similar shape and structure compared to the previously described embodiment of FIG. 4. Specifically, The V-shaped implant 126 includes a semi-rigid V-shaped member 128, which forms a compressible V shape that substantially matches the funnel shape of the uterus 104. Further, the V-shaped member is enclosed within two layers of fabric, preferably knitted PET fabric 130. Generally, the PET fabric 130 would have an open mesh structure, preferably made from 70/34 textured PET yarns and sewn together with 4-0 braided PET suture 131. In this respect, the PET fabric 130 provides improved contact with the wall of the uterus 104 when the V-shaped implant 126 is implanted.

Rolled Fabric Implant

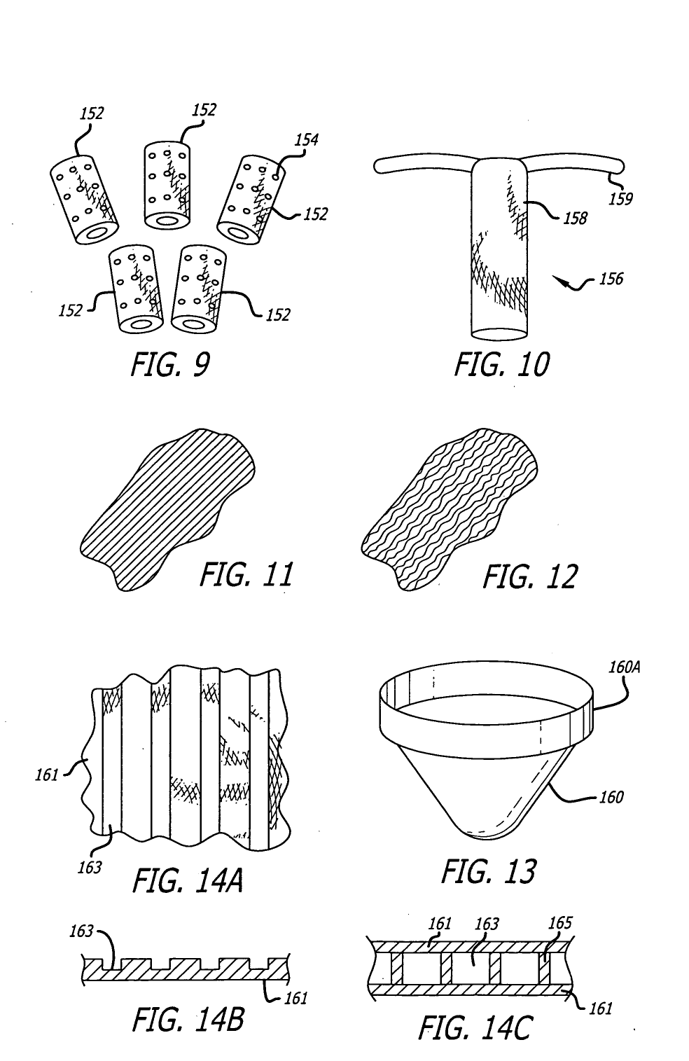

FIG. 9 illustrates rolled fabric implants 152 according to the present invention. In this preferred embodiment, multiple rolled fabric implants 152 are implanted within a uterus 104 to create a tissue response. Each fabric implant 152 is preferably about 4.5 mm in diameter and about 1.75 cm in length and is created by rolling approximately 4 layers of fabric, although dimensions may vary in length and thickness. Bard DeBakey Elastic Knit PET (Style #6106) cardiovascular fabric has been utilized in initial prototypes, but more optimal fabric constructions are contemplated by this invention. As mentioned in previous embodiments, apertures/channels 154 may be included within the fabric (preferably 1/16 inches in diameter, randomly placed 2-5 mm apart) to increase the potential for creating bridging adhesions within the uterus 104. PET sutures (not shown) along the free edge of each fabric implant 152 maintain the rolled shape of the rolled fabric implant 152.

Preferably, approximately 5 fabric implants 152 are deployed within a uterus 104 (one or two at a time) through transcervical approach involving a cannula as similarly described above. The number of rolls can be varied based on uterine size and other factors. Without a rigid or semi-rigid inner structure, the fabric implants 152 better conform to the shape of the uterus 104, creating a larger area of contact with the uterine walls. Additionally, there is no stent or other solid object that may inhibit tissue in-growth from penetrating through the device.

Rolled Fabric Implant With Stent

FIG. 10 illustrates yet another preferred embodiment according to the present invention. The implant 156 includes fabric 158, preferably 4 layers of fabric sutured to maintain an elongated roll shape. Although Bard DeBakey Elastic Knit PET (Style # 6106) was utilized in initial prototypes, other more optimal fabric constructions are contemplated by this invention. The fabric 158 may include 1/16 inch apertures randomly spaced about 2-5 mm apart to increase the potential of creating a bridging adhesive tissue growth. The fabric implant 156 includes a semi-rigid member 159 that extends in a T shape from one end of the roll of fabric 158. Optionally, this semi-rigid member 159 may be present within and extend throughout the roll of fabric 158. As described in previous embodiments within this application, the semi-rigid member 159 may, for example, be a Mirena IUD stent with the hormone cylinder removed and the center shaft optionally removed.

The fabric implant 156 may be implanted by a similar procedure as described in T-shaped implant device 100, using a cannula during a transcervical procedure. Once implanted, the fabric implant 156 creates adhesions that result in an architectural change along the centerline of the uterine cavity.

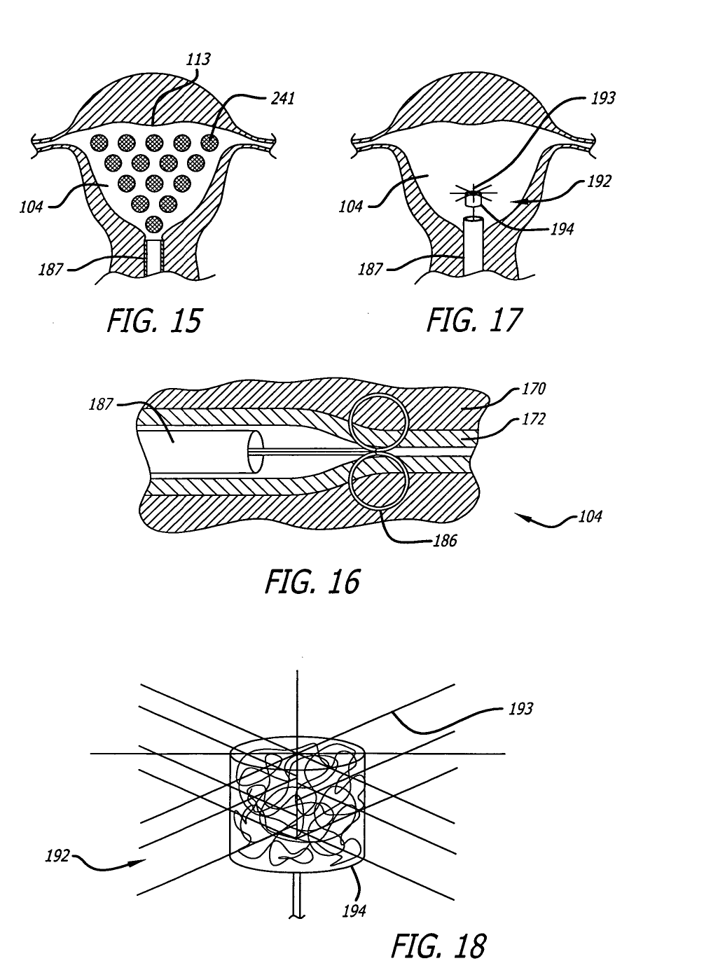

In another preferred embodiment, seen in FIG. 15, according to the present invention, a single or multiple fabric ball(s) 241 having tissue response inducing properties may be implanted within a uterus or cervix. Preferably, the fabric is composed of a PET or other material mesh, similar to the examples previously described. The compliant nature of the fabric ball 241 allows for maximum contact with the uterine tissue and/or walls, thus creating a desired tissue response that would preferably induce amenorrhea, or at least reduced bleeding.

Uterine Cervix Plug

FIG. 13 illustrates another preferred embodiment of the present invention in the form of a cervix plug 160. While intended to be implanted within the uterus, the cervix plug 160 is similar in shape to a contraceptive cervical cap, having an overall cup or cone shape. However, the cervix plug 160 is composed of flexible material such as silicone or other material that would allow the cervix plug 160 to be compressed and delivered into the uterine cavity 104 through a cannula. Once within the uterus 104, the convex end of the cervix plug 160 is oriented to face the endocervical os opening into the lower uterine segment, i.e., in a proximal direction, and a tool or suture attached to the convex end is then used to pull the cervix plug 160 proximally into the internal endocervical os 116a, causing blockage. The tool or suture is then released, allowing a rim 160a of the cervix plug 160 to prevent the plug from being ejected from the cervix 116. Ultimately, the plugged endocervix 116 results in amenorrhea, ceasing all bleeding.

In a similar preferred embodiment, a small implant (not shown) comprised of PET may be implanted into the endocervix 116 or internal cervical os 116a, causing a fibroproliferative response that totally occludes the canal and blocks access to the uterus 104. Thus, the cervical PET or other fabric implant would induce amenorrhea by a mechanism similar to the previous embodiment.

Antimicrobial Material

In another preferred embodiment of the present invention, an implant device contains an antimicrobial agent to induce tissue trauma within the uterus, killing endometrial cells and eroding down to the junction of the endometrium with the myometrium (junction) or into the myometrium, where a fibroproliferative or other tissue response can be stimulated. Since adhesion creation often requires contact with the junction or myometrium, incorporation of an antimicrobial coating may alleviate the need for pre-treatment procedures.

For example, an antimicrobial silver ion coating may be added to an implant discussed in this application by using ion beam deposition on the implant's fibers or alternately by integrating the antimicrobial into the fibers during the polymerization and extrusion process. Needle felt fabrics constructed from the fibers with impregnated silver may then be used to create an implant device with a desired shape, including but not limited to, the shapes described elsewhere in this application, for example. In this manner, the antimicrobial silver wears away, develops a zone of inhibition, retards growth or otherwise injures the endometrial tissue, allowing the fibers to stimulate and create adhesions within the uterus.

In addition to antimicrobial agents, other trauma inducing agents may be administered or eluted from an implanted device. For example, silver nitrate, tetracycline, alcohols, and other agents.

Architecture Modifying Devices

As previously described, creating a fibroproliferative or other tissue response which bridges between the uterine walls and obliterates the entire cavity remains a compelling mechanism for creating amenorrhea. However, discrete architectural changes of the uterine cavity that involves adhesions or mechanical connections that bridge between the uterine walls, may also reduce menstrual bleeding or result in amenorrhea.

The uterus is a muscle that undergoes mechanical contractions due to a variety of biological and physical stimuli. These contractions are a normally occurring event during menstruation. During a typical normal contraction, the muscle experiences a contraction/stress pattern that is transmitted between the myometrial muscle fibers in the anterior and posterior walls in a circumferential fashion. This contraction forms a pulsatile wave across the cavity. When the body is subjected to or imparts an electrical or mechanical stimulus, there is generally a feedback loop that allows the body to make physiological adjustments when necessary or to continue to respond normally if the feedback is normal.

The previously described myometrial muscle contraction may be normally expected in an unaltered uterus. However, a uterus having both the anterior and posterior walls joined together may develop additional interactions from the contracting opposite wall near or in the middle of the uterine cavity. With this interaction, contracting stress patterns and stress magnitudes may be changed during a contraction, both local to the attachment site and more globally to the entire or a distant region of the uterus. These changes may result in abnormal tissue feedback that would result in biological changes, altering and possibly stopping menstrual bleeding.

In this respect, a preferred embodiment is illustrated in FIG. 29 according to the present invention, which creates architectural changes of the uterus. A suture 174 is delivered into the uterus 104 trans-vaginally or by external laparoscopic or open abdominal/pelvic techniques. The suture 174 may be preferably composed of a polymer or metal and penetrates through the endometrium 172 and partially into the myometrium 170. Alternately, multiple sutures 174 or staples may be used, as well as other suturing or stapling tools.

In a similar preferred embodiment, the posterior and anterior walls of the uterus 104 may be joined with a biological adhesive, such as fibrin or a polymer adhesive such as cyanoacrylate. Thus, an architectural change is created within the uterus, inducing reduced bleeding and preferably amenorrhea.

FIG. 21 illustrates yet another embodiment of the present invention which creates architectural changes within the uterus 104. A tie member 180 punctures the uterine walls, passing through the endometrial and myometrial tissue layers. The tie member 180 may be a rigid, semi-rigid, or flexible cable/thread/wire member having ends that attach to fasteners 182 either within the myometrium or on the serosal surface.

When implanted, the tie member 180 creates tension between the fasteners 182, pulling the walls of the uterus 104 together, preferably near the lower or middle regions of the uterus 104. The large size of the fasteners 182 spread out the bearing load of the tie rod member 182 and inhibit tissue breakdown and pull through of the implant. The tie member 180 and fasteners 182 may be deployed through a laproscopic minimally invasive surgical approach, external to the uterine cavity. Additionally, transvaginal or open surgical procedures may also be used.

FIG. 31 illustrates another embodiment of the present invention that creates an architectural change within the uterus with a mechanical connection between the walls of the uterus 104. The barbed connector 400 contains a main strut 401 with multiple protruding barbs 402 on each opposing end of the strut. The implant length is such as to provide engagement into the myometrial tissue 404, beyond the depth of the endometrial tissue layer. The barbs are oriented to easily penetrate the endometrium 403 and myometrial wall, but resist pull-out once in place. One method of deployment is to pressurize the uterine cavity to distend the uterine walls. The barbed connector can be positioned at any location where a connection between the anterior and posterior walls is desired using an endoscopic grasper through a transcervical approach. Once in position, the distending pressure can be released and the uterine walls will collapse down upon the barbed connector and engage the barbs. The device can be made of any biocompatible polymer or metal material with the appropriate mechanical characteristics. Stainless steel or a shape memory alloy such as Nitinol are two examples of possible materials.

Although mechanical connectors have primarily been described as embodiments for architecture modifying devices, the architecture change can also be obtained by discreet adhesions between the walls. Thus, any of the embodiments within this disclosure that create a bridging adhesion between the uterine walls can result in an architecture change that can preferably result in amenorrhea or possibly reduced bleeding. This includes the T-shaped and V-shaped devices, as well as the tissue penetrating devices listed in the following paragraphs. Tissue Penetratinq Implant

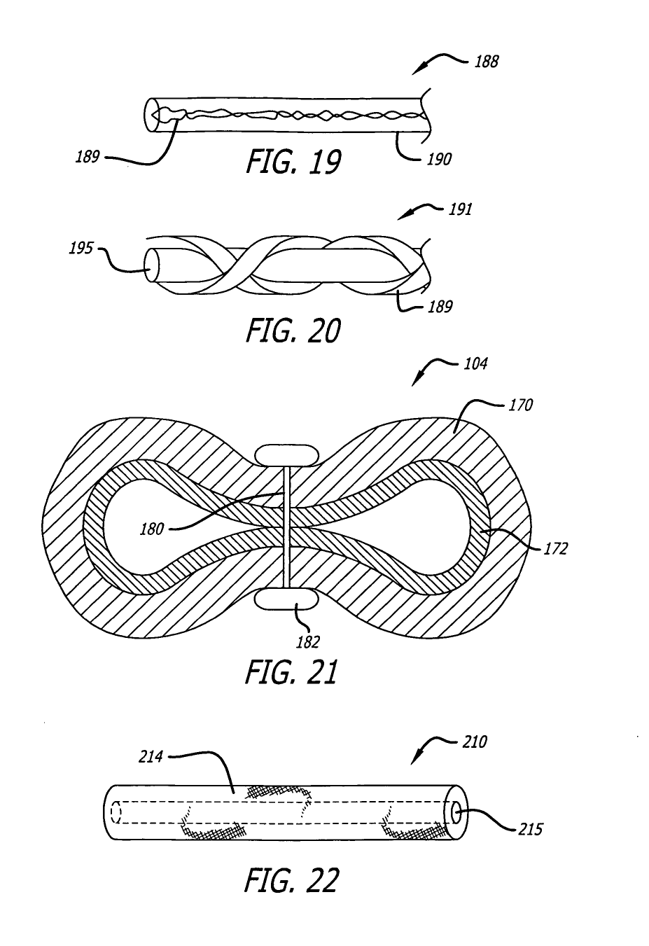

FIG. 16 illustrates another embodiment of the present invention, which includes rings 186 that penetrate through the endometrium 172 and into the myometrium 170. Each ring 186 is preferably composed of a shape-memory tube 190, such as a nitinol tube, and contains PET fibers 189 within the tube 190 as seen in FIG. 19 or around the wire 195 as seen in FIG. 20, creating a scaffold for tissue ingrowth between the walls of the uterus 104. Thus, the rings 186 cause trauma by puncturing the uterine tissue while providing the PET scaffolding for a tissue ingrowth response, such as a fibroproliferative or stroma type. In this respect, pretreatment procedures may not be necessary, since the rings 186 themselves penetrate the endometrium 172 and myometrium 170.

The shape memory tubes 186 may be deployed into the uterus 104 through a cannula 187. A distal end of the cannula 187 is placed at the target location in the uterus 104, where the change in architecture is desired from the tissue adhesion. As the shape memory tubes 186 are advanced out from the cannula 187, they bend to their pre-shaped form, curling and thus penetrating into the endometrium 172 and myometrium 170.

If the wire strut 191, seen in FIG. 20, is used for the rings 186, the PET fibers 189 (optionally braided) allow tissue growth to follow along the rings 186, bridging across the walls of the uterus 104. Similarly, if the tube strut 188, seen in FIG. 19, is used for the rings 186, the PET fibers 189 provide a path within the tube 190 for the tissue to grow on. To provide tissue access to the PET fibers 189 within the tube strut 188, the tube 190 may be perforated by laser, chemical etching, or similar perforation methods. To further enhance the adhesion formation within the uterus 104, additional PET fibers or fabric (not shown) may be placed within the uterus 104 at the deployment site of the ring 186, either before or after deployment of the rings 186.

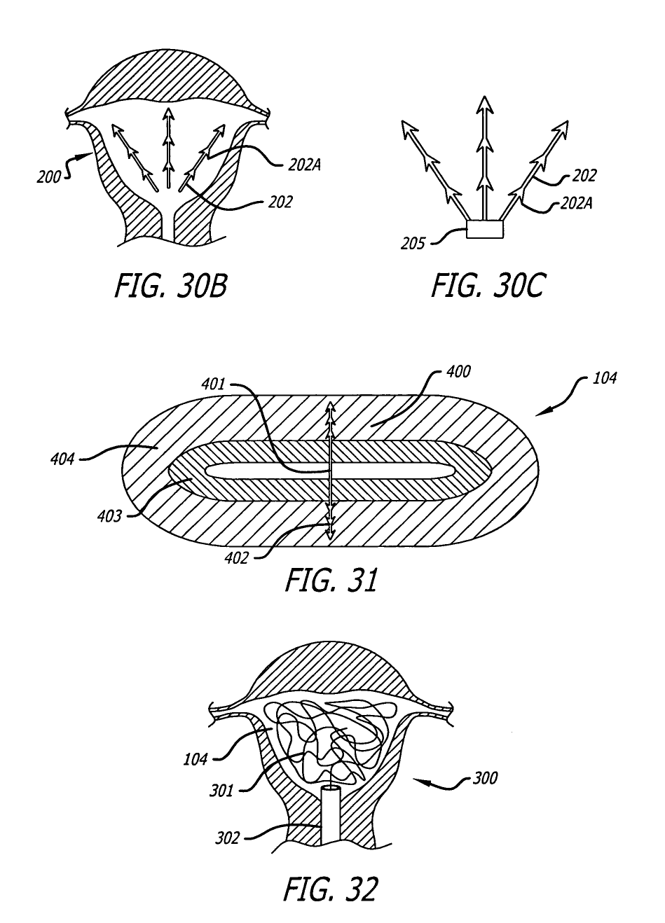

FIG. 30A illustrates another preferred embodiment of the present invention which includes branch device 200 having spines 202a protruding in multiple directions. Each spine 200a is attached to a branch 202 with multiple spines 202a. Multiple branches 200 may be deployed individually, as seen in FIG. 30B or connected to a single unit 205 as seen in FIG. 30C. The deployment can be accomplished by a simple cannula 204 accessing the uterus transcervically. As with the previous embodiment, the spines 202a may be configured as the tube strut 188 or wire strut 191, as seen in FIGS. 19 and 20 respectively. In this respect, the spines 202a are preferably composed of a shape memory material such as nitinol and further include PET 189 within the tube 190 or outside of the wire 195. Each branch 202 includes multiple spines 202a that protrude at varying points along the length of the branch 202. The branches 202 and spines 202a preferably collapse to conform within the deployment cannula and allow for positioning within a patient. Once the cannula 204 is positioned at a desired location within the uterus 104, the branch device 200 may be moved in a distal direction, allowing the branches 202 to “pop out” to the preferred configuration seen in FIG. 30. Maximum tissue penetration and engagement may be obtained by retracting the branch device 200 proximally, toward the cervix 116, forcing the spines 202a to expand away from the branches 202. Thus, the branches 202 and spines 202a allow fibrotic and/or other tissue growths to develop within the uterus 104, creating a tissue bridge and an overall change in the architecture of the uterus 104.

FIGS. 17 and 18 illustrate yet another preferred embodiment of a tissue penetrating implant. This embodiment 192 is partially bioresorbable according to the present invention, having a central member 194 with radial partially bioresorbable elements 193. The central member 194 is also composed of a bioresorbable material, such as polyglycolic acid (PGA), with intermixed PET fibers randomly oriented within the central member 194 material. The partially bioresorbable elements 193 may be similar in design to tube strut 188 or wire strut 191, seen in FIGS. 19 and 20, however the tube 190 or wire 195 is composed of a bioresorbable material such as PGA. Polyactic acid (PLA) and combinations of PGA and PLA are also potential bioresorbable substances. The bioresorbable material shall be preferably configured to provide the partially bioresorbable elements 193 with the appropriate stiffness needed to penetrate into the endometrium and myometrium.

The bioresorbable implant device 192 is delivered into the uterus 104 with a cannula 187. The implant device 192 is loaded within the cannula 187 so that the partially bioresorbable elements 193 are folded proximally, towards the user. As the central member 194 is moved out of the cannula 187 at a desired treatment location within the uterus 104, the partially bioresorbable elements 193 extend radially outward, penetrating the endometrium and myometrium layers. This penetration of partially bioresorbable elements 193 may be further enhanced by moving the implant device 192 in a proximal direction, forcing the partially bioresorbable elements 193 deeper into the tissue of the uterus 104. The central member 194 and partially bioresorbable elements 193 remain within the uterus 104, creating a fibrotic tissue response. As soon as about 2 to 4 weeks after deploying the implant device 192, the bioresorbable material begins to breakdown and resorb into the body, making the implant device 192 more compliant and likely less uncomfortable to the patient. The PET within the partially resorbable elements 193 and within the central member 194 provide the scaffold for the tissue growth and do not resorb. Additionally, since the partially bioresorbable elements 193 penetrate into the myometrium, treatment prior to the implantation procedure may not be required. In an alternative preferred embodiment, the elements 193 may be completely bioresorbable, allowing the elements 193 to penetrate into the myometrium, then completely degrade.

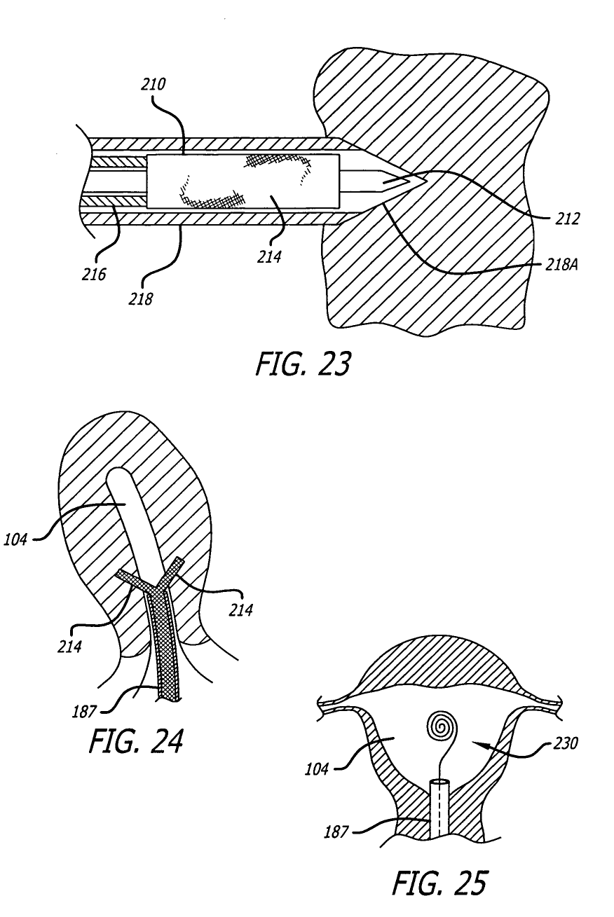

FIGS. 22-24 illustrate another preferred embodiment of a tissue penetrating implant 210 according to the present invention. The tissue penetrating implant 210 includes a fiber 214 braided with PET yarn to form an overall tubular shape with an open core or a resorbable middle core insert 215, which will generate a tissue ingrowth response when implanted within the uterus 104. In the first example, the fiber implant has an open core 215. Preferably, this fiber implant 214 is about 1-2 mm in diameter and may vary widely in length. The fiber implant 214 loads within a delivery sheath 218 having a pointed distal end 218a. The fiber implant 214 is positioned over a boring needle 212, which can be longitudinally moved in a distal or proximal direction to extend out of or into the delivery sheath 218. At the proximal end of the loaded fiber implant 214 is a tubular pushing member 216, which also fits over the boring needle 212 and extends out the proximal end of the delivery sheath 218, allowing a user to push the fiber implant 214 distally out of the delivery sheath 218.

In operation, a cannula 187 is used to position the tissue penetrating implant 210 at a desired location within the uterus 104. The pointed end 218a of the delivery sheath 218 and the boring needle 212 are advanced together into the uterine tissue 104, puncturing and penetrating the endometrium and into the myometrium.

Once the delivery sheath 218 has achieved a desired depth of penetration, a user manipulates the pushing member 216 to deploy the fiber implant 214 into the tissue. The delivery sheath 218, the needle 212, and the pushing member 216 are then retracted from the delivery site, leaving the fiber implant 214 partially within the uterine tissue 104 and partially within the uterine cavity. Thus, the PET fibers of the fiber implant 214 create a surrounding and/or ingrowth tissue response in the myometrium, as well as act as a tissue scaffold for ultimately creating a tissue bridge across the uterine cavity 104. As seen in FIG. 24, multiple fiber implants 214 may be implanted at desired target locations within the uterus 104, such as in the anterior and posterior uterine walls 104, providing additional tissue response within the myometrial tissue, ultimately inducing amenorrhea or reduced uterine bleeding.

In a similar preferred embodiment (not shown), the fiber implant may have a solid, resorbable middle (e.g. hydrophobic) probe core 215, eliminating the use of the boring needle. Instead, the pointed end of the delivery catheter solely penetrates the tissue of the uterus. Following reabsorption, the implant will have a central canal through which to propagate the tissue response associated with the implant fabric.

Contact Pressure Devices

As previously described in this application, FIG. 14C illustrates a layered fabric 161 with parallel running spacers 165 that provide separation between the fabric layers and result in open channels 163 for tissue in-growth and propagation. In an alternate embodiment if the spacers are stiff relative to the fabric, they may cause increased localized contact pressure with the uterine wall that will vary depending on the amount of uterine anterior and posterior wall separation. This local contact pressure may help to erode or traumatize the endometrial tissue and provide access to the junction or myometrial tissues. This tissue access provided by the local contact pressure may help to induce a tissue response without the need for a pretreatment. When placed in the uterus, either alone or as part of an implant device previously disclosed, the tissue response to the implant is intended to ultimately reduce bleeding and preferably induce amenorrhea. This description also applies to the embodiment of FIGS. 14A and 14B, which shows the open channels 163, however, there is no second outer layer of fabric, leaving the channels exposed for contact with the uterine wall.

In another preferred embodiment (not shown), beads may be enclosed in a fabric bag, such as a PET fabric described elsewhere in this application. Fewer larger beads will create localized contact pressure that may help focally erode or traumatize the tissues, while many smaller beads will tend to provide a more uniform contact pressure. Although the more uniform pressure may not fully erode the tissue layers, it may provide additional trauma or help inactivate the endometrium. The beads easily move within the fabric bag, allowing the bead-bag implant to conform to the shape of the uterine cavity, yet maintain contact and pressure on the uterine tissue. Thus, the pressure and contact provided by the implant will induce fibrotic or other tissue growth, ultimately reducing bleeding and preferably inducing amenorrhea.

In yet another preferred embodiment, a pressurized balloon implant (not shown) includes a compliant material, preferably being covered in a PET fabric, such as the PET fabrics disclosed elsewhere in this specification. The pressurized balloon implant is preferably inflated with liquid after implantation within the uterus. As the implant inflates, it creates contact and pressure against the uterine cavity tissue with the PET fabric. In this manner, a fibrotic or other tissue response may be induced, reducing bleeding and preferably inducing amenorrhea.

Trauma Inducing Device

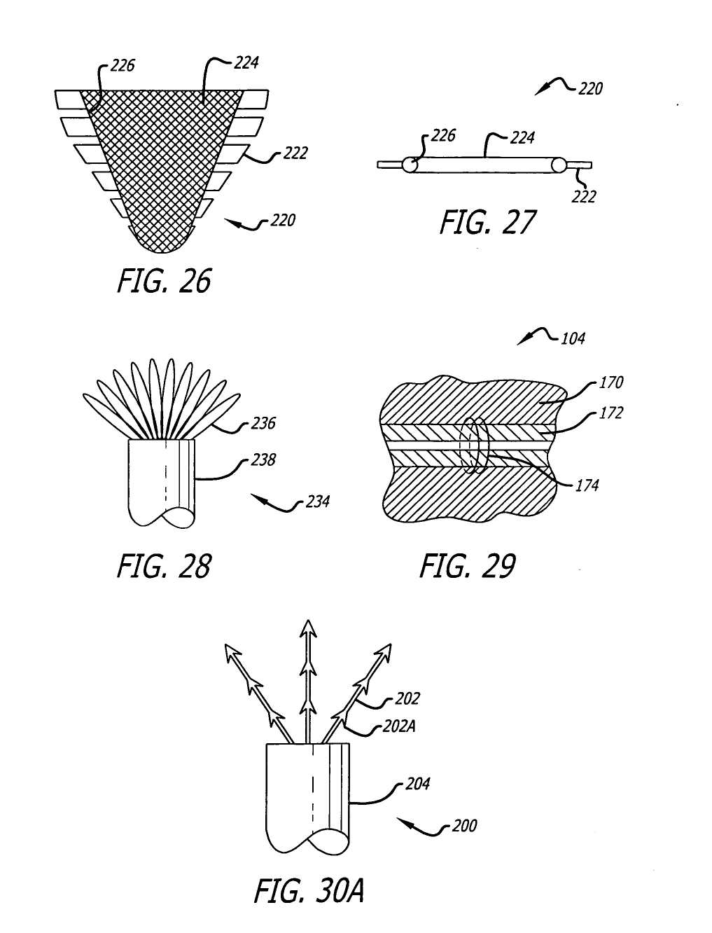

FIGS. 26 and 27 illustrate an embodiment of a trauma inducing implant 220 according to the present invention. The trauma inducing implant 220 is similar to the V-shaped implant 126 seen in FIG. 5, having a V-shaped member 226 and fabric 224 positioned between the edges, filling the center, of the V-shaped member 226. However, cutting members 222 are included, fixed longitudinally along the V-shaped member 226. The cutting members 222 cut into or traumatize tissue adjacent to the fabric 224. This produces an endometrial trauma and pushes the implant down to the junction area where a durable tissue response and in-growth can be initiated. The trauma inducing implant 220 may include one or multiple sets of the cutting member 222, each of which is preferably composed of stiff material that cuts into the tissue at desired positions. Thus, the trauma inducing implant 220 may deactivate the endometrial tissue by cutting through the functionalis layer of the endometrium and contacting basalis layer endometrial or myometrial junction. This cutting may provide access to the appropriate tissues to encourage tissue ingrowth onto the fabric 224, which acts as a tissue scaffold, without the need for pretreatment.

FIG. 25 illustrates a trauma-inducing device 230 according to the present invention. Preferably, the device 230 is composed of a strip of metal, polymer or bioresorbable polymer that has a pre-configured coil shape and sharp edges. The material may be composed of a shape memory material such as Nitinol so that it can tolerate large deformations necessary for cannula deployment, but capable of returning to a predeformed shape after deployment. The device 230 may be straightened within a cannula 187 for loading within a uterus 104. As the device 230 is advanced out of the cannula 187 within the uterus 104, it assumes its pre-configured coil shape. Once fully implanted, the device 230 scrapes across the endometrial surface, penetrating to the endomyometrial surface. The device may be wrapped with PET or other similar material that provides a tissue supportive scaffold and generates a tissue response.

Fluid Delivery

In another preferred embodiment of the present invention (not shown), tissue response inducing fibers, such as PET, may be suspended within a fluid, and then injected/pumped into the uterine cavity. The fluid may then be slowly removed from the uterus, leaving the tissue response inducing fibers within the uterus. Alternately, these fibers may also be suspended in a fluid, which can harden or solidify once pumped into a uterus, allowing the tissue response inducing fibers such as PET to contact the uterine walls and cause a tissue response.

Continuous Fiber

In another preferred embodiment seen in FIG. 32, a continuous fiber implant 300 that has tissue response inducing properties may be used to illicit a tissue response and ultimately cause amenorrhea. The continuous fiber 301 may be composed of PET, a metal thread covered with PET (as seen in FIG. 20), or another polymer capable of inducing the desired tissue response. In one embodiment, the continuous fiber implant is a flowable material that substantially fills a portion of the uterine cavity or the entire uterine cavity. The flowable fiber material substance provides a scaffold for ingrowth resulting from the desired tissue response. Metal fibers covered with PET may be preferable, allowing flexibility to avoid patient discomfort, yet enough resiliency to hold it in place against the uterine tissue and provide desired tissue access. This continuous fiber may be fed into the uterus 104, or applied to the uterine walls through a delivery cannula 302 for maximum tissue contact.

Fan Devices

FIG. 28 illustrates a preferred embodiment of a fan implant 238 according to the present invention. Elongated fiber loops 236 are fixed to a stent 238 so as to fan out, matching the overall funnel shape of the uterus. This allows the fiber loops 236 to better contact a large area of uterine tissue. The fiber loops 236 are preferably made from PET, but may also be composed of PET covered fibers or other tissue response inducing materials. The stent 238 may be configured to be removed after deployment or alternatively remain in place to support the elongated fiber loops 236 in place. As with previously described embodiments within this application, the fan implant 238 may be implanted within the uterus by a cannula, oriented with the “fan” or “fan-like” shape distal to the user.

Pretreatment and Tissue Access

As previously described in this application, amenorrhea may be induced through a variety of methods, mostly requiring the generation of a durable tissue response, such as fibrotic tissue within the uterus. Durable tissue is differentiated from non-durable tissue due to its capability in resisting some level of separation or shearing force from the uterine wall and/or breakdown, reabsorption or shedding. The endometrium has macroscopic jelly-like properties, lacking resistance to shearing forces that would be tolerated by a durable tissue. Hence, it is desired to expose the implant devices to tissue near the endomyometrial junction or myometrium (about 1 mm or more below the junction) in order to obtain a durable tissue response. When the myometrium is exposed to the implant, the tissue in-growth and adhesions are the result of a fibroproliferative response that generates granulation tissues with resulting collagen deposition, a response similar to wound healing or tissue repair. When the tissue near the endomyometrial junction is exposed to the implant, adhesions are created that consist of an aglandular histiocytic and/or stromal appearing tissue of mesenchymal origin. Some of the implants disclosed herein are designed to contact the aforementioned tissues by their very designs. However, other designs may require pre-treatment of the uterus prior to implantation to optimally generate the desired tissue response.

Endometrial resection is one pretreatment method according to the present invention, involving the complete or partial removal of both the functionalis and basalis endometrium with a variable thickness of inner myometrium. Common methods of achieving endometrial resection include the use of electrosurgical loops or roller ball devices. The endometrium is accessed trans-cervically with the patient potentially under general anesthesia. These methods cut away the endometrium with each pass of the surgical instrument with coagulation of the new surface created in the uterine cavity. If high temperatures or energy levels are used, the newly exposed tissues may be thermally fixed which would potentially block direct contact with viable myometrial tissue and prevent the desired tissue response.

Complete resection may be an effective standalone treatment for AUB, since generally previously reported amenorrhea rates as high as 50% can be obtained. The use of an implant, as described herein, would allow for increased amenorrhea rates, higher than for resection alone. Alternately, a partial resection could be used with an implant device, reducing the invasiveness and skill required to perform an effective procedure.

Endometrial ablation is another pretreatment method according to the present invention which is similar to resection. Endometrial ablation is intended to destroy all endometrial layers and a portion of the inner myometrium within the uterine cavity by a variety of ablation tools, such as high temperature circulating water, low temperature freezing probes, microwaves, RF resistance heating, and chemicals. It may be possible to use one or more of these techniques to achieve the desired myometrial tissue exposure prior to implant insertion, however any dead tissue created by these techniques should preferably be removed from the uterine cavity and endocervical canal before inserting an implant device.

Generally, endometrial ablation methods can be standalone treatments for AUB, often having effectiveness similar or slightly better than resection. Additionally, ablation is easier to perform on a patient and is potentially less invasive. These ablation techniques, in combination with a uterine implant, may significantly improve patient outcomes over ablation alone.

Dilation and curettage (D&C) is another pretreatment according to the present invention which includes dilation of the cervix and mechanical scraping of the endometrium to remove the functionalis layer of the endometrium, variable basalis endometrium and potentially some superficial myometrium. D&C is less penetrating into the uterine tissue than resection or ablation but is generally not as effective of a treatment for AUB alone; since the procedure does not remove as much endometrium and its junction with the myometrium. However, it may be possible to reach some areas of the endomyometrial junction with a more aggressive curettage, especially when the endometrium is thinnest after menstruation. D&C may be an effective pretreatment prior to inserting a uterine implant, especially if the device design takes advantage of the potentially variable and non-uniform exposure of the junction or myometrium, such as though an abrasive, pressure or other mechanism.

Cycle timing is another pretreatment according to the present invention where various drugs are administered to a patient to provide consistent menstrual cycle timing at the time of implant insertion. This timing allows an implant to be implanted within a patient when the endometrial tissue is at a desired thickness. For example, if the implant device is to be implanted when the endometrial tissue thickness is at a minimum, Zoladex, birth control or a similar cycle controlling drug could be utilized to synchronize the menstrual cycle and allow for implant insertion at a specific point in the menstrual cycle.

Hormones are another pretreatment method according to the present invention. Such hormonal pretreatment can be used alone or in combination with other pretreatment methods, including other pretreatments discussed herein. In such pretreatments, hormones are injected just prior to, at the time of, or after implanting a uterine device to enhance or direct a tissue response. In one example, estrogen may be used to produce a fibrinolysis effect within the uterus. In another example, progesterone may be injected into a patient, which may promote signaling that leads to a fibrous response within the uterus, especially when used with a uterine implant. Although progesterone is commonly used for hormone therapy to cause menstruation, it is always given during the secretory phase of the endometrial cycle when estrogen levels within a patient are high. However, progesterone is not commonly given earlier in the cycle (e.g. early proliferative phase), which could stimulate adhesions through the generation of tissues such as collagen. Thus, early progesterone pretreatment during the menstrual cycle, in combination with an uterine implant may increase the outcome of inducing amenorrhea.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof. What is claimed is:

1. An intrauterine implant comprising:
a body member having an elongated body and at least two arm members fixed near an end of said body member; and
a scaffold material disposed on said body member, said scaffold material formulated to react with uterine tissue so as to substantially inhibit uterine bleeding.

2. An intrauterine implant according to claim 1, wherein said scaffold material is PET.

3. An intrauterine implant according to claim 1, wherein said scaffold material has a triangular shape as disposed on said body member.

4. An intrauterine implant according to claim 1, wherein said scaffold material encapsulates said body member.

5. An intrauterine implant according to claim 1, wherein said scaffold material includes adhesion bridging apertures interspersed along a surface of said material.

6. An intrauterine implant according to claim 5, wherein said adhesion bridging apertures are approximately 1/16 inch in diameter.

7. An intrauterine implant according to claim 1, further comprising a fallopian tube extension structure extending from each of said at least two arms; said fallopian tube extension structure being sized to extend from each of said at least two arms towards an opening of fallopian tubes of a uterus.

8. An intrauterine implant according to claim 1, further comprising a cervical extension structure from said elongated body; said cervical extension structure being sized to extend from said elongated body into a cervix.

9. An intrauterine implant according to claim 1, wherein said scaffold material is a fabric material.

10. An intrauterine implant according to claim 9, wherein said scaffold material is one of a knit mesh, a woven mesh, and a non-woven mesh.

11. An intrauterine implant according to claim 1, wherein said body member is comprised of a polymer.

12. An intrauterine implant according to claim 1, wherein said body member is comprised of a metal.

13. An intrauterine implant according to claim 7, wherein a scaffold material is disposed on said fallopian tube extension structure.

14. An intrauterine implant according to claim 8, wherein a scaffold material is disposed on said cervical extension structure.

15. An intrauterine implant according to claim 1, wherein said scaffold material is formulated to cause the formation of adhesions.

16. An intrauterine implant according to claim 1, wherein said scaffold material is formulated to cause the formation of fibrosis.

17. An intrauterine implant according to claim 1, wherein said scaffold material has a pore size between fibers of approximately 50 to 250 micrometers.

18. An intrauterine implant according to claim 17, wherein said scaffold material has a pore size of approximately 100-200 micrometers.

19. An intrauterine implant comprising:
a body member having two arm members connected to form an overall V-shape;
a scaffold material spanning a space between said two arm members; said scaffold material formulated to react with uterine tissue so as to substantially inhibit uterine bleeding.

20. An intrauterine implant according to claim 19, wherein said scaffold material is comprised of randomly oriented yarn fibers.

21. An intrauterine implant according to claim 19, wherein said scaffold material is comprised of a layer of open mesh structure.

22. An intrauterine implant according to claim 19, wherein said scaffold material is comprised of PET.

23. An intrauterine implant according to claim 21, wherein said scaffold material is comprised of two layers.

24. An intrauterine implant according to claim 20, wherein said scaffold material has a pore size between yarn fibers of approximately 50 to 250 micrometers.

25. An intrauterine implant according to claim 19, wherein said arm members are configured to be resiliently compressible between a compressed delivery state to an expanded deployed state, said expanded deployed state being of sufficient size so as to match said implant with the intrauterine cavity shape.

26. An intrauterine implant according to claim 19, wherein said scaffold material is a fabric material.

27. An intrauterine implant according to claim 26, wherein said scaffold material is one of a knit mesh, a woven mesh, and a non-woven mesh.

28. An intrauterine implant according to claim 19, wherein said body member is comprised of a polymer.

29. An intrauterine implant according to claim 19, wherein said body member is comprised of a metal.

30. An intrauterine implant according to claim 19, wherein said scaffold material is formulated to cause the formation of adhesions.

31. An intrauterine implant according to claim 19, wherein said scaffold material is formulated to cause the formation of fibrosis.

32. An intrauterine implant according to claim 19, further comprising at least one pressure member disposed on at least one of said arm members and extending away therefrom, said at least one pressure member being sized and shaped to create a pressure point on a uterine wall following implantation of said body in a uterus.

33. An intrauterine implant according to claim 32, wherein said at least one pressure member includes a plurality of pressure members being disposed on each of said two arm members.

34. An intrauterine implant according to claim 32, wherein at least one pressure member is a cutting member with sufficient sharpness for said cutting member to cut into at least the endometrium aspect of the myometrium of said uterus.

35. An implant for reducing intrauterine bleeding comprising:
at least one cylindrically shaped roll of fabric material;
said at least one cylindrically shaped roll of scaffold material sized and shaped for positioning in a uterus;
said scaffold material being formulated to react with uterine tissue so as to substantially inhibit uterine bleeding.

36. An implant according to claim 35, wherein said scaffold material is PET.

37. An implant according to claim 35, wherein said implant includes a plurality of cylindrically shaped rolls of scaffold material.

38. An implant according to claim 35, wherein each of said at least one cylindrically shaped roll of scaffold material is about 2-4.5 mm in diameter and about 1.75 cm in length.

39. An implant according to claim 35, wherein said at least one cylindrically shaped roll of scaffold material has approximately four layers of scaffold material.

40. An implant according to claim 35, wherein said scaffold material includes a plurality of apertures for promoting bridging adhesions.

41. An implant according to claim 35, wherein said scaffold material is a fabric material.

42. An implant according to claim 41, wherein said scaffold material is one of a knit mesh, a woven mesh, and a non-woven mesh.

43. An implant according to claim 35, wherein said scaffold material is formulated to cause the formation of adhesions).

44. An implant according to claim 35, wherein said scaffold material is formulated to cause the formation of fibrosis.

45. An implant according to claim 35, wherein said scaffold material has a pore size of approximately 50 to 250 micrometers.

46. An intrauterine implant comprising:
a body member having an elongated body and at least two arm members extending substantially laterally from one end of said elongated body;
said elongated body comprising a scaffold material; and, said scaffold material being formulated to react with uterine tissue so as to substantially inhibit uterine bleeding.

47. An intrauterine implant according to claim 46, wherein said elongated body is a cylindrical roll of said scaffold material.

48. An intrauterine implant according to claim 47, wherein said cylindrical roll of scaffold material includes approximately four layers of scaffold material.

49. An intrauterine implant according to claim 46, wherein said elongated body further includes a structural member extending within said scaffold material.

50. An intrauterine implant according to claim 49, wherein said structural member is a semi rigid member.

51. An intrauterine implant according to claim 46, wherein said scaffold material includes a plurality of apertures for promoting bridge adhesions.

52. An intrauterine implant according to claim 46, wherein said scaffold material is a fabric material.

53. An intrauterine implant according to claim 52, wherein said scaffold material is one of a knit mesh, a woven mesh, and a non-woven mesh.

54. An intrauterine implant according to claim 46, wherein said body member is comprised of a polymer.

55. An intrauterine implant according to claim 46, wherein said body member is comprised of a metal.

56. An intrauterine implant according to claim 46, wherein said scaffold material is formulated to cause the formation of adhesions.

57. An intrauterine implant according to claim 46, wherein said scaffold material is formulated to cause the formation of fibrosis.

58. An intrauterine implant according to claim 46, wherein said scaffold material has a pore size of approximately 50 to 250 micrometers.

59. An implant to reduce uterine bleeding comprising:
a cone shaped plug member;
said plug member being comprised of a resiliently deformable material;
said plug member having a maximum diameter such that said plug member frictionally engages an internal endocervical os of a patient when released from a deformed state and thereby retains said plug member in position in said internal endocervical os.

60. An implant according to claim 59, wherein said resiliently deformable material is silicone.

61. An implant according to claim 59, wherein said resiliently deformable material is a material that is formulated to react with uterine tissue.

62. An implant according to claim 61, wherein said resiliently deformable material is PET.

63. An intrauterine implant comprising:
a scaffold material sized and shaped to fit within a uterus of a patient;
a plurality of depressions disposed on one face of said scaffold material;
said scaffold material being formulated to react with uterine tissue so as to substantially inhibit uterine bleeding.

64. An intrauterine implant according to claim 63, wherein said plurality of depressions includes a plurality of parallel spacers.

65. An intrauterine implant according to claim 63, wherein said scaffold material is PET.

66. An intrauterine implant according to claim 63, wherein said depressions include a plurality of rectangularly shaped depressions.

67. An intrauterine implant according to claim 64, wherein said parallel spacers are comprised of a material harder than said scaffold material so as to provide localized contact surfaces against uterine tissue.

68. An intrauterine implant according to claim 63, further comprising a second layer of scaffold material for overlay onto said scaffold material and to thereby enclose said depressions.

69. An intrauterine implant according to claim 63, wherein said scaffold material has a pore size of approximately 50 to 250 micrometers.

70. An implant for reducing intrauterine bleeding comprising:
a plurality of generally spherical members;
said generally spherical implants sized and shaped for placement within a uterus of a patient;
said generally spherical implants being formulated from a material that reacts with uterine tissue so as to substantially inhibit uterine bleeding.

71. An implant according to claim 70, wherein said spherical members are comprised of a scaffold material.

72. An implant according to claim 71, wherein said scaffold material is PET.

73. An implant according to claim 70, wherein said scaffold material is a fabric material.

74. An implant according to claim 73, wherein said scaffold material is one of a knit mesh, a woven mesh, and a non-woven mesh.

75. An implant according to claim 70, wherein said scaffold material is formulated to cause the formation of adhesions.

76. An implant according to claim 70, wherein said scaffold material is formulated to cause the formation of fibrosis.

77. An implant for reducing intrauterine bleeding comprising:
a resiliently deformable ring member having a substantially circular relaxed state and a substantially linear stressed state;
at least one end of said deformable ring member having a sharp point for penetration into a myometrium of a uterus of a patient;
a scaffold material disposed on said ring;
said scaffold material being formulated for reacting with uterine tissue so as to substantially inhibit uterine bleeding.

78. An implant according to claim 77, wherein said ring member is comprised of Nitinol.

79. An implant according to claim 77, wherein said scaffold material is PET.

80. An implant according to claim 77, wherein said scaffold material is disposed internal to said ring.

81. An implant according to claim 80, wherein said ring includes a plurality of apertures disposed on said ring thereby exposing said scaffold material to tissue ingrowth in said uterus.

82. An implant according to claim 77, wherein said scaffold material is disposed on an external surface of said ring.

83. An implant according to claim 77, wherein said scaffold material has a pore size of approximately 50 to 250 micrometers.

84. An intrauterine implant comprising:
an implant body;
a scaffold material disposed on said implant body, said scaffold material being formulated to react with uterine tissue and thereby inhibit uterine bleeding;
a plurality of struts extending away from said implant body a sufficient amount so as to cause injury to uterine tissue when said implant body is positioned in a uterus.

85. An intrauterine implant according to claim 84, wherein said implant is at least partially bioresorbable.

86. An intrauterine implant according to claim 84, wherein said struts are bioresorbable.

87. An intrauterine implant according to claim 86, wherein said struts are comprised of a bioresorbable material sufficiently strong to penetrate into at least an endometrium of said uterus.

88. An intrauterine implant according to claim 87, wherein said bioresorbable material is PGA.

89. An intrauterine implant according to claim 84, wherein said struts contain said scaffold material.

90. An intrauterine implant according to claim 89, wherein said scaffold material on said struts is PET.

91. An intrauterine implant according to claim 84, wherein said scaffold material is a fabric material.

92. An intrauterine implant according to claim 91, wherein said scaffold material is one of a knit mesh, a woven mesh, and a non-woven mesh.

93. An implant according to claim 84, wherein said scaffold material is formulated to cause the formation of adhesions.

94. An implant according to claim 84, wherein said scaffold material is formulated to cause the formation of fibrosis.

95. An implant according to claim 84, wherein said plurality of struts extend a sufficient amount so as to contact myometrial tissue.

96. An implant according to claim 84, wherein said scaffold material has a pore size of approximately 50 to 250 micrometers.

97. A uterine device for inhibiting bleeding in a uterus comprising:
a tie member extendable between opposing walls of said uterus; and
a fastening mechanism disposed on said tie member such that said tie member may be constrained so as to bring said opposing walls of said uterus into close proximity to each other.

98. A uterine device according to claim 97, wherein said tie member is rigid.

99. A uterine device according to claim 97, wherein said tie member is semi-rigid.

100. A uterine device according to claim 97, wherein said opposite ends of said tie member are sufficiently sharp so as to puncture uterine tissue of said opposing walls of said uterus.

101. A uterine device according to claim 97, wherein said fastener is sized to distribute load caused by said tie member causing contact of said opposing inner walls of said uterus.

102. A uterine device according to claim 97, wherein said tie member is a plurality of sutures.

103. A uterine device according to claim 102, wherein said fastening mechanism is a knot.

104. A uterine implant assembly comprising:
a sheath having a lumen;
an implant disposed within said lumen of said sheath;
a boring mechanism operably associated with said implant;
a pushing member disposed in said lumen of said sheath behind said implant; said pushing member actuatable to move said implant out of said sheath and into uterine tissue to a sufficient degree so as to cause injury to said uterine tissue.

105. A uterine implant assembly according to claim 104, wherein said implant is substantially cylindrical in shape.

106. A uterine implant assembly according to claim 104, wherein said implant is comprised of a braided scaffold material.

107. A uterine implant assembly according to claim 106, wherein said braided scaffold material is PET.

108. A uterine implant assembly according to claim 105, wherein said substantially cylindrical implant further includes an open core.

109. A uterine implant assembly according to claim 105, wherein said substantially cylindrical implant further includes a core probe.

110. A uterine implant assembly according to claim 109, wherein said core probe is bioresorbable.

111. A uterine implant assembly according to claim 104, wherein said boring mechanism is a boring needle disposable within said sheath and movable in and out of said sheath.

112. A uterine implant assembly according to claim 104, wherein said boring mechanism comprises a sharp point disposed on one end of said implant.

113. A uterine implant assembly according to claim 104, wherein said pushing member is sized and shaped to move said implant into said uterine tissue to a sufficient degree so as to contact myometrial tissue.

114. A uterine implant for inhibiting uterine bleeding within a uterus comprising:
a member comprised of shape memory material movable between a normal state and a formed state;
said normal state being a substantially coiled shape;
said formed state being a substantially planar elongated shape suitable for placement of said member in a delivery cannula;
said member having sharp edges so as to cause injury to uterine tissue as said member changes from said formed state to said normal state within said uterus.

115. A uterine implant according to claim 114, wherein said member is wrapped in a scaffold material formulated to react with uterine tissue so as to inhibit uterine bleeding.

116. A uterine implant according to claim 115, wherein said material is PET.

117. A uterine implant according to claim 114, wherein said sharp edges are sufficiently sharp so as to penetrate at least the endometrial tissue of said uterus.

118. A uterine implant according to claim 115, wherein said scaffold material is a fabric material.

119. A uterine implant according to claim 118, wherein said scaffold material is one of a knit mesh, a woven mesh, and a non-woven mesh.

120. A uterine implant according to claim 114, wherein said scaffold material is formulated to cause the formation of adhesions.

121. An implant according to claim 114, wherein said scaffold material is formulated to cause the formation of fibrosis.

122. An intrauterine implant comprising:
a stent having a first end and a second end; and
a plurality of loops disposed on one of said first and second ends of said stent;
said plurality of loops sized and shaped to substantially conform to a shape of said uterus;
said plurality of loops being comprised of a scaffold material to react with uterine tissue so as to substantially inhibit uterine bleeding in said uterus.

123. An intrauterine implant according to claim 122, wherein said scaffold material is PET.

124. An intrauterine implant according to claim 122, wherein said plurality of loops is removably disposed on said stent.

125. An intrauterine implant according to claim 122, wherein said scaffold material is a fabric material.

126. An intrauterine implant according to claim 125, wherein said scaffold material is one of a knit mesh, a woven mesh, and a non-woven mesh.

127. An intrauterine implant according to claim 122, wherein said scaffold material is formulated to cause the formation of adhesions.

128. An intrauterine implant according to claim 122, wherein said scaffold material is formulated to cause the formation of fibrosis.

129. An intrauterine implant according to claim 122, wherein said scaffold material has a pore size of approximately 50 to 250 micrometers.

130. An intrauterine implant comprising:
an elongated body member; and
a plurality of angled spines disposed on said elongated body member;
said spines being movable between a compressed state when said elongated body member is disposed in a delivery device and an extended state when said elongated body member is located in a uterus;
said spines being sized and shaped to cause tissue injury when said elongated body is located in said uterus.

131. An intrauterine implant according to claim 130, wherein each spine includes a scaffold material formulated to react with uterine tissue so as to substantially inhibit uterine bleeding.

132. An intrauterine implant according to claim 131, wherein said scaffold material is disposed in an internal bore of at least one spine.

133. An intrauterine implant according to claim 131, wherein said scaffold material is disposed on an external surface of at least one spine.

134. An intrauterine implant according to claim 130, wherein said intrauterine implant includes multiple elongated body members being interconnected to each other.

135. An intrauterine implant according to claim 134, wherein said intrauterine implant comprises three elongated body members, each of which being connected to each other at proximal end of said elongated body member.

136. An intrauterine implant according to claim 131, wherein said scaffold material is a fabric material.

137. An intrauterine implant according to claim 136, wherein said scaffold material is one of a knit mesh, a woven mesh, and a non-woven mesh.

138. An intrauterine implant according to claim 131, wherein said scaffold material is formulated to cause the formation of adhesions.

139. An intrauterine implant according to claim 131, wherein said scaffold material is formulated to cause the formation of fibrosis.

140. An intrauterine implant according to claim 130, wherein said spines are disposed on each of opposite ends of said elongated member.

141. An intrauterine implant according to claim 130, wherein said spines are disposed substantially along the length of said elongated member.

142. An intrauterine implant comprising:
a fiber thread;
said fiber thread being comprised of a scaffold material formulated to react with uterine tissue so as to substantially inhibit uterine bleeding;
said fiber thread having a length allowing substantial coverage of the inside of a uterus.

143. An intrauterine implant according to claim 142, wherein said fiber thread is comprised of a metal thread covered with fabric material.

144. An intrauterine implant according to claim 143, wherein said fabric material is PET.

145. An intrauterine implant according to claim 142, wherein said fiber thread is a metallic.

146. An intrauterine implant according to claim 145, wherein said metallic fiber thread is sufficiently flexible so as to avoid causing patient discomfort and sufficiently stiff so as to ensure proper contact between said fiber thread and said uterine tissue.

147. An intrauterine implant according to claim 142, wherein said scaffold material is a fabric material.

148. An intrauterine implant according to claim 147, wherein said scaffold material is one of a knit mesh, a woven mesh, and a non-woven mesh.

149. An intrauterine implant according to claim 142, wherein said scaffold material is formulated to cause the formation of adhesions.

150. An intrauterine implant according to claim 142, wherein said scaffold material is formulated to cause the formation of fibrosis.

151. An intrauterine implant according to claim 142, wherein said fiber thread is comprised of a flowable biocompatible polymer that solidifies within the intrauterine cavity so as to obtain substantial coverage of the intrauterine cavity and contact with desired uterine tissues.

152. An intrauterine implant according to claim 151, wherein said flowable biocompatible polymer is PET.

153. An intrauterine implant assembly comprising:
a deployment body member having an elongated body and at least two arm members disposed at one end of said body member;
a scaffold material having a pair of pockets for removably receiving said at least two arm members;
a delivery cannula;
said deployment body member being movable into and out of said cannula;
said scaffold material formulated to react with uterine tissue so as to substantially inhibit uterine bleeding.

154. An intrauterine implant according to claim 153, wherein said scaffold material is PET.

155. An intrauterine implant according to claim 153, wherein said scaffold material has a triangular shape as deployed by said deployment body member.

156. An intrauterine implant according to claim 153, wherein said scaffold material includes adhesion bridging apertures interspersed along a surface of said material.

157. An intrauterine implant according to claim 156, wherein said adhesion bridging apertures are approximately 1/16 inch in diameter.

158. An intrauterine implant according to claim 153, wherein said scaffold material is a fabric material.

159. An intrauterine implant according to claim 158, wherein said scaffold material is one of a knit mesh, a woven mesh, and a non-woven mesh.

160. An intrauterine implant according to claim 153, wherein said deployment body member is comprised of a metal.

161. An intrauterine implant according to claim 153, wherein said scaffold material is formulated to cause the formation of fibrosis.

162. An intrauterine implant according to claim 153, wherein said scaffold material has a pore size between fibers of approximately 50 to 250 micrometers.

163. An intrauterine implant according to claim 162, wherein said scaffold material has a pore size of approximately 100-200 micrometers.

164. An intrauterine implant according to claim 153, further including a removable suture extending from said delivery cannula to said scaffold material.

165. A method of deploying an uterine implant comprising:
providing a deployment stent movably mounted within a delivery cannula;
providing a scaffold material;
removably locating said scaffold material onto said deployment stent;
urging said scaffold material toward a fundic wall of a uterine cavity;
expanding said scaffold material;
removing said deployment stent from said scaffold material;
retracting said deployment stent into said delivery cannula.

166. A method according to claim 165, wherein the urging of said scaffold material toward a fundic wall includes tensioning said scaffold material with a suture extending to said user.

167. A method according to claim 166, the suture is removed from the scaffold material prior to removing said deployment stent.

168. A method according to claim 165, wherein the providing of said scaffold material includes providing a PET material.

169. A method according to claim 165, wherein the providing of said scaffold material includes providing a triangular fabric material.
